(19) United States
(12) Patent Application Publication
(10) Pub. No.: US 2012/0231063 A1
(43) Pub. Date: Sep. 13, 2012

(54) DRESSING COMPRISING ACTIVE COMPONENTS OF CENTELLA ASIATICA AND USE OF THE SAME

(75) Inventors: Chun-Hsu YAO, Taichung City (TW); Jen-Yu Yeh, Taichung City (TW)

(73) Assignee: CHINA MEDICAL UNIVERSITY, Taichung City (TW)

(21) Appl. No.: 13/161,882

(22) Filed: Jun. 16, 2011

(30) Foreign Application Priority Data
Mar. 11, 2011 (TW) 100108274

(51) Int. Cl. A61L 15/20; A61L 15/44; A61K 36/185; B29C 47/00; A61L 15/40; A61K 31/704; B82Y 5/00

(52) U.S. Cl. 424/445; 514/33; 424/725; 264/465; 977/788; 977/906

(57) ABSTRACT

Provided is a dressing, comprising (a) a substrate layer and (b) an active layer, which is a nanofiber layer and comprises: (b1) at least one of gelatin and collagen; (b2) polyvinyl alcohol (PVA); and (b3) at least one of asiaticoside and a Centella asiatica extract. Also, a method for preparing the dressing is provided.

### CROSS-REFERENCES TO RELATED APPLICATIONS

This application claims the benefit of Taiwan Patent Application No. 100108274, filed on Mar. 11, 2011 in the Taiwan Intellectual Property Office, the disclosure of which is incorporated herein in its entirety by reference.

### FIELD

The present invention relates to a dressing comprising an active component of Centella asiatica and use thereof. In particular, the present invention relates to the use of the dressing in wound healing.

### BACKGROUND

As the complexity of human life and work increases, it is inevitable that individuals in daily life may suffer from traumas, such as abrasions, cuts, surgical wounds, or even severe burns or scalds. A wound dressing is covered on the wound when there is a wound or defect on the tissue or organ to temporarily replace the epidermis or endothelial tissue and protect the wound from infection by external bacteria or dirty substances and to promote wound healing, thereby letting damaged tissues restore their functions. Because the causes of wounds are complicated, conventional wound dressings, like gauze, cotton or cotton pads, can no longer manage the caring for various types of wounds. In addition, as the demand for medical treatment quality increases, a wound dressing that can provide a better healing environment, reduce the number for dressing changes, decrease infection, improve patients' life quality, etc., is required.

To meet the aforesaid demand, various dressings have been developed, including antibacterial dressings, algin dressings, foam, silicone hydrogel, hyaluronic acid dressings, etc. However, the dressings on the market still have many defects. For example, for a wound with more exudates (such as pus or blood), it would require frequent dressing changes when the exudates are secreted continuously. The dried exudates often make a dressing adhere to the wound, which results in secondary damage to the wound and damage of nascent tissues surrounding the wound when the dressing is removed for dressing changes. The wound is thus hard to heal and wound inflammation may even arise, causing pain and discomfort to the patients. In addition, because the air permeability of a dressing is poor, the wound is not easy to heal and may suffer from bacterial infection. These conventional dressings also often cannot provide a satisfactory drug release rate, and cannot achieve the effective treatment effect because the release rate is too slow. At times, the release rate of these dressings is too fast, and drugs therein leak out too early and the dressings become invalid accordingly. Patients therefore must frequently change the dressings before the wound heals, resulting in more pain and inconvenience to the patients. Furthermore, some drugs under high concentration may generate cytotoxicity to cells, which decreases the rate of wound healing. At this point, it is more practical to avoid rapid drug release rate models. Therefore, a dressing that can improve the above defects is still in demand in the market.

The present invention is a result of the study conducted for the above demands, and provides a dressing with good biocompatibility and drug release rate. The dressing has a nanofiber layer comprising an active component of Centella asiatica, polyvinyl alcohol (PVA), and at least one of gelatin and collagen.

### SUMMARY

The primary objective of the present invention is to provide a dressing, comprising:

(a) a substrate layer; and

(b) an active layer, which is a nanofiber layer and comprises:
(b1) at least one of gelatin and collagen;
(b2) polyvinyl alcohol (PVA); and
(b3) at least one of asiaticoside and a Centella asiatica extract.

Another objective of the present invention is to provide a method for preparing the above dressing.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram showing the double layer structure of the dressing of the present invention;

FIG. 2 is a schematic diagram showing the electrospinning equipment for preparing the dressing of the present invention;

FIG. 3 is a statistical bar diagram showing the effect of the concentration of a Centella asiatica extract liquid on fibroblast cell growth;

FIG. 4 is an SEM diagram of the dressing of the present invention;

FIG. 5 is an SEM diagram of an EGC (electrospinning gelatin nanofiber combined with Centella asiatica) active layer containing no Centella asiatica extract liquid;

FIG. 6 is an SEM diagram of the EGC active layers prepared from electrospinning solutions comprising different volume ratios of the Centella asiatica extract liquid;

FIG. 7 is a statistical bar diagram showing the effect of the EGC active layers prepared under different electrospinning operation times on fibroblast cell growth;

FIGS. 8A to 8C are HPLC chromatograms showing the concentration of asiaticoside released by the EGC active layers prepared from electrospinning solutions containing different concentrations of the Centella asiatica extract liquid;

FIG. 9 is a curve diagram showing the degradation rate of the EGC active layer;

FIG. 10 is a statistical bar diagram showing the swelling degree of the EGC active layer;

FIG. 11 is a statistical bar diagram showing the water contact angle of the EGC active layer; and

FIG. 12 is a comparison photo showing the wound healing effect of various dressings on a rat.

### DESCRIPTION OF THE PREFERRED EMBODIMENT

Unless otherwise stated herein, the terms “a (an)”, “the” or the like used in this specification (especially in the Claims hereinafter) shall be understood to encompass both the singular form and the plural form, and the term “nanofiber” refers to fiber with an average diameter less than 1000 nanometers. In addition, for clearance, in the attached figures, the size of each element and area may be exaggerated and is not drawn to scale.

Centella asiatica has functions of promoting wound healing of tissues, enhancing learning and memorizing abilities, improving peripheral blood circulation, reducing lower limb edema, improving peptic ulcer and gastrointestinal inflammation, regulating immune function, improving skin quality, anti-tumor, etc. Centella asiatica contains asiaticoside, which can promote the proliferation of fibroblast cells and speed up wound healing. Asiaticoside has the structure of the following Chemical Formula (I):

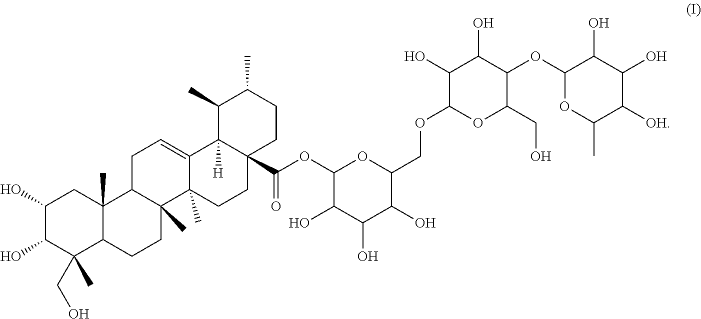

The present invention combines an active component of Centella asiatica to provide a dressing, comprising:

(a) a substrate layer; and

(b) an active layer;

wherein the active layer is a nanofiber layer and comprises:

(b1) at least one of gelatin and collagen;

(b2) polyvinyl alcohol (PVA); and

(b3) at least one of asiaticoside and a Centella asiatica extract.

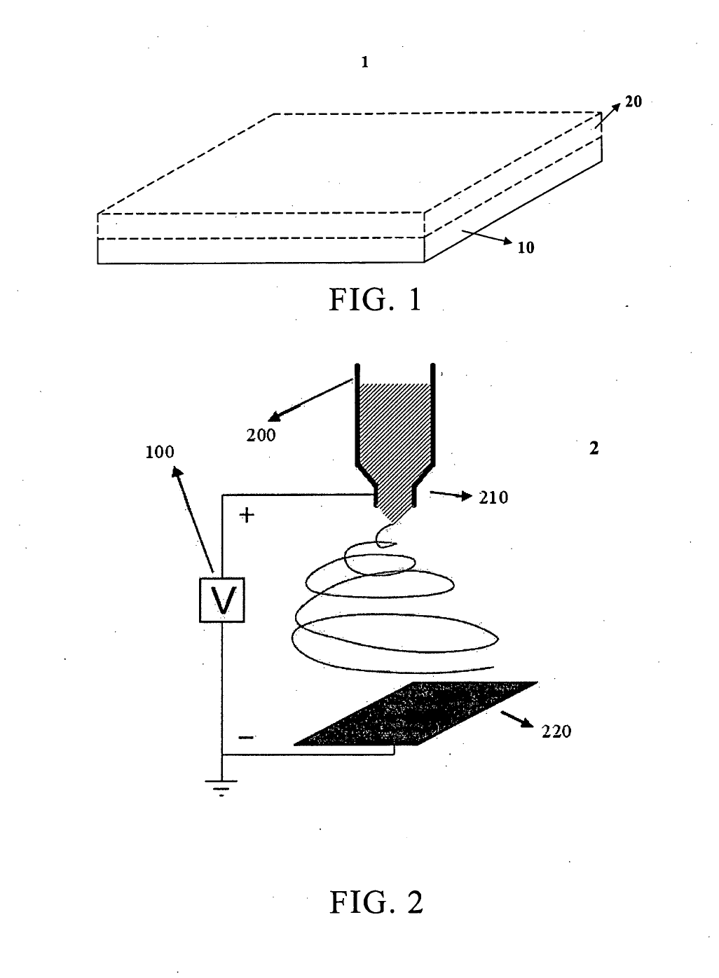

FIG. 1 illustrates an embodiment of the structure of the dressing of the present invention; wherein, the dressing 1 has a double layer structure, comprising a substrate layer 10 and an active layer 20. The active layer 20 has a nanofiber structure, and when the dressing 1 is used, the active layer 20 is the layer that is in contact with the skin.

In the dressing 1, any materials that can block bacteria can be used to form the substrate layer 10. The material is preferably biodegradable. For example, the substrate layer 10 is a chitosan layer. Chitosan is a biodegradable material and has good biocompatibility, and the amino groups in its structure under an acidic condition become positively charged NH³⁺ groups, which can interfere with the negative charges on the bacterial surface to change the cell wall's permeability, letting substances within the bacteria flow outside to cause cell death. In addition, after small chitosan molecules enter bacteria cells, they can reduce the bacterial vitality by complexing with DNA and affecting the chromosome structure, and thus achieve the bacteria inhibition effect. Therefore, the substrate layer 10 provided by chitosan, apart from supporting the active layer 20 to provide the desired supportive function, can prevent bacteria from passing through the dressing to infect the tissue or wound.

The active layer 20 is composed of nanofiber, and the average diameter of the fiber is less than 1000 nm, preferably less than 500 nm, more preferably less than 200 nm. The nanofiber comprises: (b1) at least one of gelatin and collagen; (b2) polyvinyl alcohol (PVA); and (b3) at least one of asiaticoside and a Centella asiatica extract. Preferably, in the active layer 20, the amount of ingredient (b1) is about 80% to 99% based on the total weight of ingredients (b1) and (b2).

Because the active layer 20 has a nanofiber structure, it has high air permeability (or porosity) and a large specific surface area, and can increase air circulation to the wound tissue and increase the contact area between the wound tissue and the medicament (such as asiaticoside) in the active layer 20. Thus, the proliferation of fibroblast cells is promoted and the fibroblast cells are induced to enter the dermis layer and secret a large amount of the extracellular matrix (such as collagen, growth factors, hematopoietic factors, etc.) to achieve the effects of fast wound healing, reducing the chance of bacterial infection, and leaving no scars to speed up tissue repair, regeneration and wound recovery.

Furthermore, the active layer 20 uses gelatin and/or collagen as the substrate ingredients. Gelatin is a substance produced by the degradation or denature of natural collagen. These two materials (i.e., gelatin and collagen) will not generate repulsion to the skin when coming into contact with the skin and also can be degraded by the body fluid, and hence, they have excellent biocompatibility. Therefore, when the dressing 1 is used, the active layer 20 can be degraded by the tissue and absorbed while assisting in wound recovery to avoid tissue damage or the secondary damage of the wound caused by tearing off the dressing, thereby, reducing patients' pain suffering. Moreover, gelatin and collagen have structure and functions similar to the natural extracellular matrix, and can promote the adhesion and proliferation of fibroblast cells to achieve the effects of accelerating wound healing and shortening treatment period.

In an embodiment of the present invention, the active layer 20 of the dressing 1 is formed onto a surface of the substrate layer 10 by electrospinning. For example, the active layer 20 is formed by the following steps:

(I) providing a first solution comprising ingredient (b1);

(II) providing a second solution comprising ingredient (b2);

(III) mixing the first solution and the second solution to provide a third solution;

(IV) adding a fourth solution comprising ingredient (b3) into the third solution to obtain a fifth solution;

(V) electrospinning the fifth solution to form a fiber layer onto a surface of the substrate layer; and

(VI) carrying out a crosslink reaction to crosslink the fiber layer to form the active layer.

In step (I), an appropriate solvent can be adopted to dissolve ingredient (b1) in the solvent to provide the first solution. For example, formic acid, acetic acid, ethanol or combinations thereof can be used. In an embodiment of the present invention, formic acid is used as the solvent.

In step (II), PVA can be dissolved in a suitable solvent to provide the second solution. For example, water can be used as the solvent.

The fourth solution in step (IV) is a solution comprising ingredient (b3) (i.e. the active component in the dressing). For example, the fourth solution can be a Centella asiatica extract liquid or an aqueous solution comprising asiaticoside. In an embodiment, a Chinese herb known as Centella asiatica and purchased from a Chinese herbal shop is directly extracted with a methanol (90% by volume) aqueous solution, and the soluble part is collected and dried and then dissolved in the water to provide the fourth solution.

FIG. 2 illustrates the operation of electrospinning and shows the scheme of an equipment 2 for carrying out electrospinning. As shown in FIG. 2, the electrospinning equipment 2 comprises a high voltage generator 100 and an injector 200 that connects to the anode of the high voltage generator 100. There is a metal needle head (or nozzle) 210 at one end of the injector 200. A target object (i.e. the substrate layer) 220 is placed at the cathode during electrospinning, and a polymer solution is placed in the injector 200. As the solution is injected to the electrified metal needle head (or nozzle) 210, the high voltage produces liquid in a pyramidal shape at the periphery of the needle head 210, and the liquid is then attracted by the electric field to move to the surface of the target object 220 at the cathode below the needle head at a certain distance. When the solution is sprayed, within a millionth of a second, the electric field will align polymer molecules to form fiber filaments. The sprayed polymer becomes nanofibers after being extended. Because electrospinning can quickly and directly transform a polymer material into nanofibers, and these formed nanofibers are in the form of a porous thin film, the nanofiber layer (not shown) formed on the surface of the target object 220 has the advantage of a large specific surface area. The relevant principle and operation of electrospinning can be seen in Frenot et al., Polymer nanofibers assembled by electrospinning, Current Opinion in Colloid and Interface Science, 8 (2003), 64-75, which is incorporated hereinto by reference.

In step (III), the above first solution and second solution are mixed to provide a third solution. Then, in step (IV), the third solution is mixed with the above fourth solution to provide a fifth solution for carrying out the electrospinning of step (V).

To make the provided active layer have a good nanofiber structure to give excellent filament formation quality, air permeability and specific surface area, it is preferable to formulate the content of polymer ingredients (i.e., ingredients (b1) and (b2)) in the fifth solution to let the solution have enough viscosity to prevent the content of the polymer molecules from being too low to spin out an uniform nanofiber layer. It is preferred for the third solution to be formulated in step (III) to make the concentration of ingredient (b1) be about 130 to 170 mg/mL and the concentration of ingredient (b2) be about 5 to 30 mg/mL. And, in step (IV), the amount of the third solution is about 60% to 95%, based on the total volume of the third solution and the fourth solution. More preferably, in the third solution of step (III), the concentration of ingredient (b1) is about 140 to 160 mg/mL, and the concentration of ingredient (b2) is about 5 to 15 mg/mL; and in step (IV), the amount of the third solution is about 80% to 90%, based on the total volume of the third solution and the fourth solution.

In step (V), the electrospinning equipment as shown in FIG. 2 can be used, in which the fifth solution is introduced into the injector, and the injector is used to carry out electrospinning on the substrate layer to form a fiber layer. The operation conditions for the electrospinning are as follows: the electrospinning is operated for about 2 to 8 hours at conductions including a voltage from about 20 to 30 KV; the feeding flow rate of the fifth solution in the injector is about 0.005 to 0.015 mL/hour; and the distance between the needle head of the injector and the surface of the substrate layer is about 7 to 13 cm. Preferably, the operation conditions for the electrospinning are as follows: the electrospinning is operated for about 3 to 5 hours at the conduction that the voltage is about 24 to 26 KV; the feeding flow rate of the fifth solution in the injector is about 0.008 to 0.012 mL/hour; and the distance between the needle head of the injector and the surface of the substrate layer is about 9 to 11 cm.

In a specific embodiment of the present invention, the below operation conditions are used to carry out electrospinning: the voltage is about 25 KV; the feeding flow rate of the fifth solution in the injector is about 0.01 mL/hour; the distance between the needle head of the injector and the surface of the substrate layer is about 10 cm; and the electrospinning operation time is about 4 hours. As shown in the following examples, the active layer prepared under the above electrospinning operation conditions has excellent filament formation quality, air permeability and specific surface area. Moreover, because in the present invention, the active component of Centella asiatica is mixed with polymer molecules and then nanofiber is produced from the mixture, the surface area of the active component of Centella asiatica can be significantly increased to improve its bioavailability.

As mentioned above, in terms of the dressing quality, controlling the release rate of a drug in a dressing is also one of the important factors. If the drug release rate is too slow, then the dressing cannot achieve the effective treatment. On the other hand, it usually takes four to eight weeks for a wound to heal completely. If the drug release rate is too fast and the drug completely runs off too early, resulting in dressing failure, then the frequency to change the dressing before the wound heals must increase, which will make patients feel inconvenient and uncomfortable. In addition, some drugs under high concentration can generate cytotoxicity to cells, thus reducing the wound healing rate instead. Under this circumstance, excessively high drug release rate should be avoided. To this end, when the dressing of the present invention is prepared, a crosslink reaction of step (VI) is further carried out to make the nanofiber layer crosslink to form a crosslinked structure to maintain and elevate the strength, rigidity, and stability of the fiber structure in the active layer, so the active component of Centella asiatica will not be released too fast due to a high degradation rate of the fiber structure, thereby achieving the effect of controlling the drug release rate.

In step (VI), for example, an aqueous solution containing glutaraldehyde of about 40 to 60% by volume can be used as a crosslinker to carry out a steam crosslink reaction on the fiber layer for about 35 to 55 minutes. Preferably, an aqueous solution containing glutaraldehyde of about 45 to 55% by volume is used to carry out a steam crosslink reaction on the fiber layer for about 40 to 50 minutes. In an embodiment, an aqueous solution containing glutaraldehyde of 50% by volume is used to carry out a steam crosslink reaction on the fiber layer for 45 minutes. As shown in the later examples, the active layer after the crosslink treatment, whether the drug concentration therein is high or low, can maintain a certain drug release rate and effectively promote wound healing, and therefore has a better wound healing effect than the dressings on the market.

Because the dressing of the present invention can provide good drug release rate and the active component of Centella asiatica contained therein has the effect of improving skin quality, promoting the proliferation of fibroblast cells, speeding up wound healing, etc, it can be used in various applications, for example, medical supplies (such as medical gauze, bandage, or fiber) or beauty products (such as a beauty mask), etc.

The present invention also provides a method for preparing the dressing, which comprises the above steps (I) to (VI). The method of the present invention has the advantages of a simple process equipment, easy procedure, convenient operation, low cost, etc.

The detailed technology and preferred embodiments implemented for the present invention are described in the following paragraphs; however, the scope of the present invention is not limited thereby.

### EXAMPLE 1

### Cell Test of a Centella asiatica Extract Liquid

Dried Centella asiatica (purchased from a Chinese herbal shop) was ground to powder. One gram of the powder was dissolved in 20 mL of a solvent (90% by volume of methanol dissolved in water) and stirred at room temperature for 5 hours, and was filtered by a filter paper. The filtrate was concentrated and dried by air extraction under reduced pressure to obtain a Centella asiatica extract. The extract was then dissolved in water to formulate an extract liquid with an extract concentration of 2500 μg/mL.

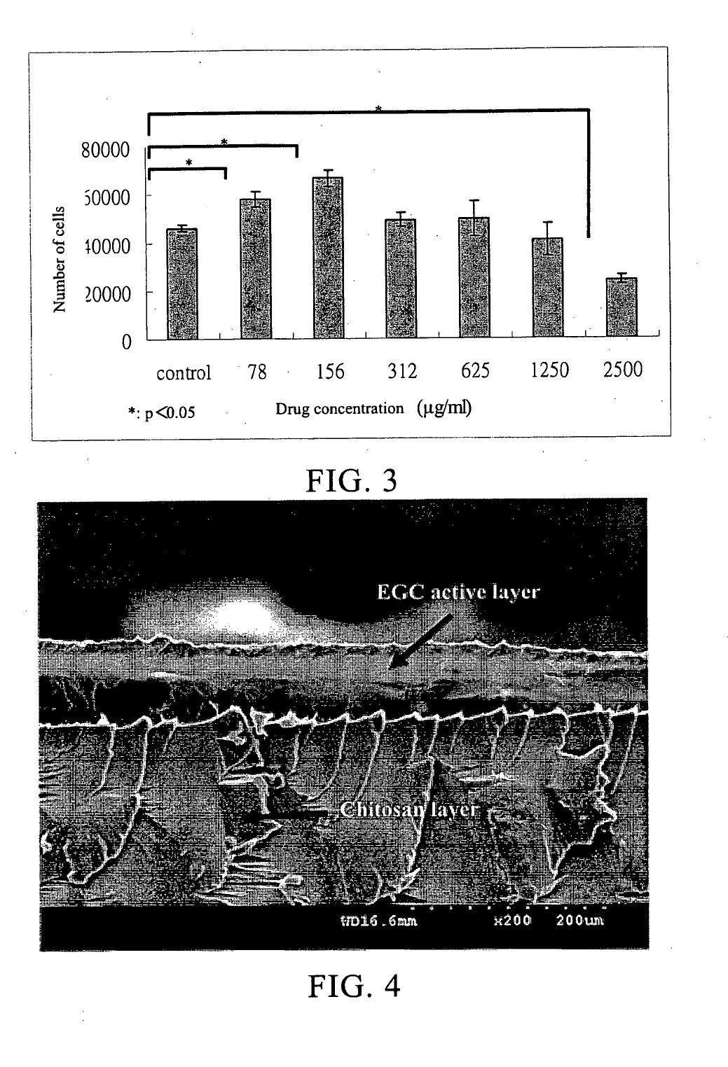

The Centella asiatica extract liquid with an original concentration of 2500 μg/mL was half diluted into six samples with different concentrations (78, 156, 312, 625, 1250, or 2500 μg/mL), and fibroblast L929 (purchased from Food Industry Research and Development Institute, Taiwan) was cultured with these samples, and then an MTT (3-(4,5)-dimethylthiahiazo(-z-yl)-3,5-di-phenytetrazoliumromide, commercial name: Thiazolyl Blue) test was carried out. The test results are shown in FIG. 3.

FIG. 3 shows that the fibroblast cells had the best proliferation rate under the concentration of 156 μg/mL of the Centella asiatica extract liquid, and when the concentration was above 312 μg/mL, then the proliferation rate decreased. Compared with the control group, the drug concentration at 2500 μg/mL achieved the most significant inhibition effect.

### EXAMPLE 2

### Preparation of a Dressing Comprising a Centella asiatica Extract Liquid

[Preparation of a Centella asiatica Extract Liquid]

Dried Centella asiatica (purchased from the Chinese herbal shop) was ground to powder. One gram of the powder was dissolved in 20 mL of a solvent (90% by volume of methanol dissolved in water) and stirred at room temperature for 5 hours, and was filtered by a filter paper. The filtrate was concentrated and dried by air extraction under reduced pressure to obtain a Centella asiatica extract. Then 312 mg of the extract was weighted by a microbalance, and was re-dissolved in 1 mL of deionized water to obtain a quantitative Centella asiatica extract liquid (i.e., a fourth solution was prepared).

### [Electrospinning]

(1) Formulating a Solution for Electrospinning

Formic acid (10 mL) was first poured into a 100 mL beaker and stirred, and then 1.7 g of gelatin powder (purchased from Sigma Chemical Co., US) was poured slowly into formic acid under stir, and the beaker was sealed with aluminum foil. The solution was then stirred for another 20 minutes (i.e., a first solution was prepared). Next, 50 mL deionized water was poured into another 500 mL large beaker and placed on a hot plate and heated to 70° C. Then, 10 mL of deionized water was poured into another 100 mL small beaker. One gram of polyvinyl alcohol (PVA, purchased from SHOWA, Showa level 1, reagent grade, Japan) was weighted and added into the small beaker, and then the small beaker was placed in the large beaker for heating, and the solution was automatically stirred for 30 minutes (i.e., a second solution was prepared).

One milliliter of the above gelatin solution was sucked and discarded, and then 1 mL of the above PVA solution was added in the gelatin solution and stirred sufficiently for 1 hour (i.e., a third solution was prepared). One milliliter of the gelatin/PVA solution was sucked and discarded, and then 1 mL of 312 mg/mL of the Centella asiatica extract liquid was added to the gelatin/PVA solution and stirred evenly for 1 hour (i.e., a fifth solution was prepared).

(2) Preparing a Chitosan Layer

Two grams of chitosan powder (purchased from Sigma Chemical Co., US) was added to 20 mL acetic acid and dissolved and then evenly mixed. After 980 mL of deionized water was added into the mixture and stirred for 24 hours, the resultant solution was poured into a glass plate container and placed in a fume chamber to form a film, and then a chitosan layer in the form of a thin film was prepared.

(3) Conducting Electrospinning

The equipment for electrospinning is shown in FIG. 2. Ten milliliters of the mixed solution (i.e., the fifth solution) of gelatin/PVA/the Centella asiatica extract liquid prepared from the above steps was introduced in a 10 mL syringe (or injector) 200, and the syringe was connected to a catheter and linked to a micro propeller (not shown), and the other end of the syringe was connected to a metal needle head 210. A high voltage electrode (+) was linked to the metal needle head 210, and the feeding flow rate of the propeller was set, and then the chitosan layer (the substrate layer 220) prepared from the above step (2) was placed on a ground collection plate (−). Then, a high voltage power supply was triggered (be aware of all possible conductive materials nearby) to carry out electrospinning, and electrospun fibers were collected. The electrospinning was operated under the following conditions: the voltage was 25 KV; the feeding flow rate of the mixture solution in the syringe was 0.01 mL/hour; the distance between the needle head 210 of the syringe 200 and the surface of the chitosan layer 220 was 10 cm; and the electrospinning was conducted for 4 hours. An electrospun nanofiber layer in which gelatin and the active component of Centella asiatica were combined was prepared.

The nanofiber layer was cut to round plate specimens with a size of 20 mm diameter, and 50% by volume of glutaraldehyde (dissolved in water) was used to conduct a steam crosslink reaction on the nanofiber layer for 45 minutes. The nanofiber layer was placed on a petri dish with a diameter of 8 cm, and then placed in a fume chamber for 1 hour to evaporate glutaraldehyde completely. A dressing comprising the active component of Centella asiatica of the present invention was prepared, and the dressing included an active layer (electronspinning gelatin nanofiber combined with Centella asiatica, hereinafter abbreviated as an “EGC active layer”). A scanning electron microscope (SEM) was used to observe the morphology of the EGC active layer. The result is shown in FIG. 4.

FIG. 4 shows a double layer structure formed by electrospinning the EGC active layer on the chitosan surface. By the filament formation mechanism of the electrospinning, the upper layer of the EGC active layer was observed to adhere on the surface of the chitosan layer. The thickness of the EGC active layer was calculated as about 40±5 microns, and the lower layer was the chitosan layer, and the thickness thereof was about 300±35 microns.

### EXAMPLE 3

### The Effect of the Ratio of the Centella asiatica Extract Liquid on the EGC Active Layer

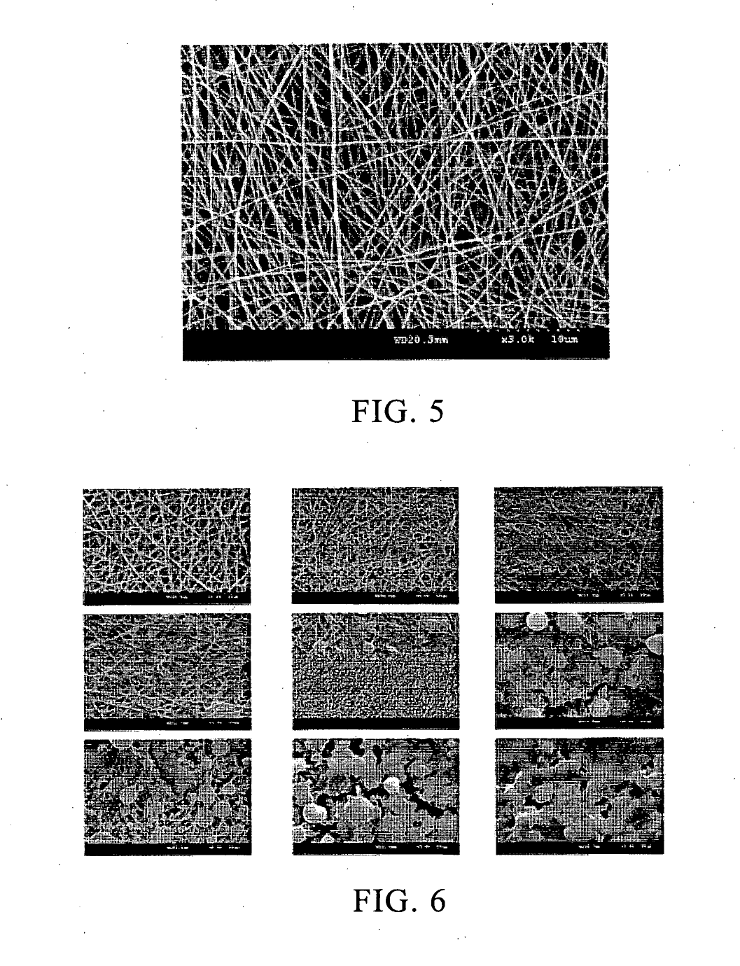

FIG. 5 is a SEM photo showing the EGC active layer that comprises no Centella asiatica extract liquid, wherein the volume ratio of the components in the solution for electrospinning was gelatin: PVA=9:1. The fiber, after statistical analysis, was calculated to have an average diameter of about 150 to 350 nm. From the MTT test in Example 1, it showed that the best concentration for the Centella asiatica extract liquid was 156 μg/mL. The Centella asiatica extract liquid with this concentration was added to solutions for electrospinning with different volume ratios to carry out electrospinning, wherein the volume ratios were the gelatin/PVA solution (gelatin/PVA=9/1): the Centella asiatica extract liquid=9:1, 8:2, 7:3, 6:4, 5:5, 4:6, 3:7, 2:8, and 1:9. The surface of the prepared EGC active layers was observed by SEM, and the morphologies are shown in FIG. 6 (A to I each was the EGC active layer added with 10%, 20%, 30%, 40%, 50%, 60%, 70%, 80%, and 90% of the Centella asiatica extract liquid).

As can be seen in FIG. 6, when the ratio of the gelatin/PVA solution and the Centella asiatica extract liquid was 6:4; the surface of the spun fiber had many water drops, indicating that the solution did not have enough viscosity, resulting in poor filament formation quality of electrospinning. When the ratio of the gelatin/PVA solution and the Centella asiatica extract liquid was 9:1, a better EGC active layer with uniform fibers can be spun out.

### EXAMPLE 4

### The Effect of Electrospinning Operation Time on the EGC Active Layer

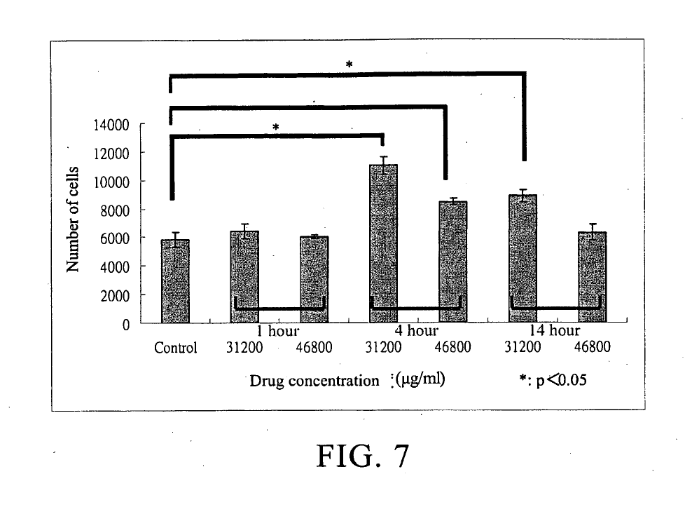

The concentration of 156 μg/mL of the Centella asiatica extract liquid was magnified by 200 and 300 times (i.e., 31,200 and 46,800 μg/mL), and based on the same method in Example 2, EGC active layers comprising different concentrations of the Centella asiatica extract liquid were prepared, and the electrospinning operation time was 1, 4 or 14 hours. Then, L929 fibroblast cells were cultured together with the prepared EGC active layers, and the number of the cells was observed. The results are shown in FIG. 7.

From FIG. 7, it can be seen that the EGC active layer prepared under the electrospinning operation time of 1 hour has no significant proliferative effect on the fibroblast cells. Under the electrospinning operation time of 14 hours, the EGC active layer with a 200-times magnified concentration of the Centella asiatica extract liquid had a proliferative effect on the fibroblast cells whereas the EGC active layer with a 300-times magnified concentration generated cytotoxicity due to excessively high drug concentration. In addition, the fibroblast cells had significant proliferation under the condition that the EGC active layer was prepared under the electrospinning operation time of 4 hours. It was estimated that under the condition that a drug concentration of 31,200 μg/mL was used to conduct electrospinning for 4 hours, the effect of the Centella asiatica extract liquid contained in the provided EGC active layer was closer to the effect of the drug concentration of 156 μg/mL in the in vitro test.

### EXAMPLE 5

### Analysis of the Drug Release Rate of the EGC Active Layer

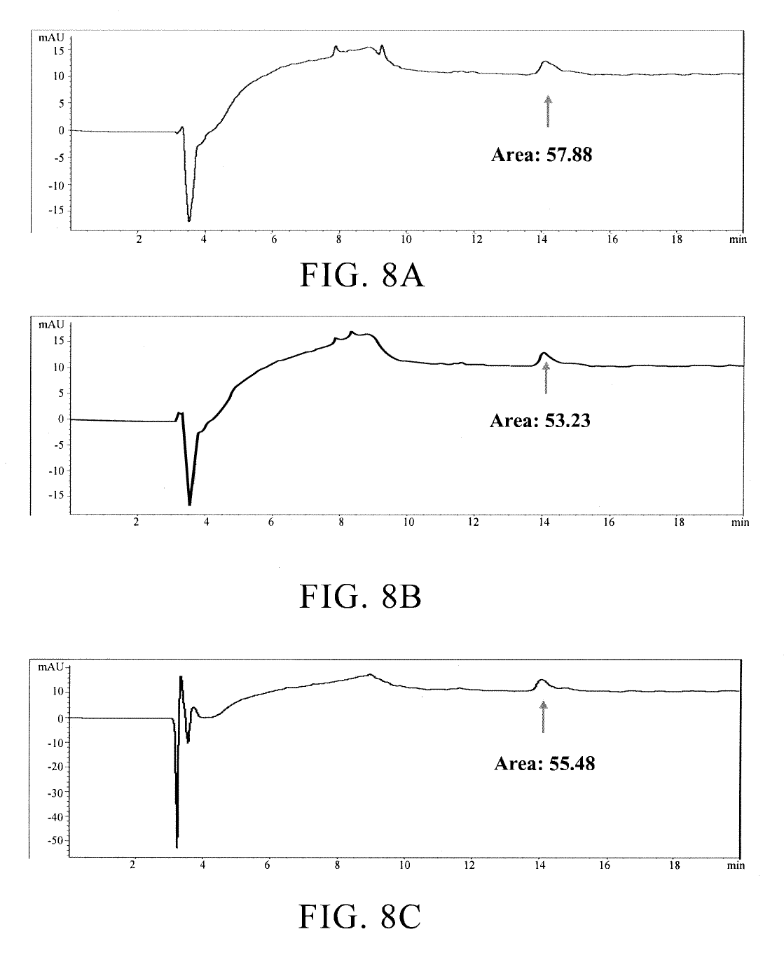

To confirm whether the EGC active layer, after being added with the Centella asiatica extract liquid, can constantly release the active component of Centella asiatica at a certain rate, the EGC active layer was immersed in the deionized water for a day in this test, and high performance liquid chromatography (HPLC) was used to measure the amount of asiaticoside in the content of the immersing solution of the EGC active layer. The results are shown in FIGS. 8A to 8C. The peak area of asiaticoside in the HPLC chromatogram was measured, and it showed that in the first day, the integrated area of asiaticoside released by the EGC active layer with the drug concentration of 46,800 μg/mL was 57.88 (FIG. 8A). The integrated area of asiaticoside released by the EGC active layer with the drug concentration of 31,200 μg/mL was 53.23 (FIG. 8B). The integrated area of asiaticoside released by the EGC active layer with the drug concentration of 156 μg/mL was 55.48 (FIG. 8C).

The test result shows that the EGC active layer prepared by the Centella asiatica extract liquid with 200 or 300-times magnified drug concentration had the same drug release rate as the EGC active layer prepared by the Centella asiatica extract liquid with the best drug concentration (i.e., 156 μg/mL) obtained in the in vitro cell test, and could release the same asiaticoside concentration. This indicates that the dressing of the present invention can effectively control drug release, and apart from effectively releasing the active component to carry out treatment, it can also avoid the adverse effect that cytotoxicity generates, the cell proliferation is inhibited, and cell repairing cannot be achieved accordingly due to excessive drug release concentration.

### EXAMPLE 6

### The Degradation Rate and Swelling Degree of the EGC Active Layer

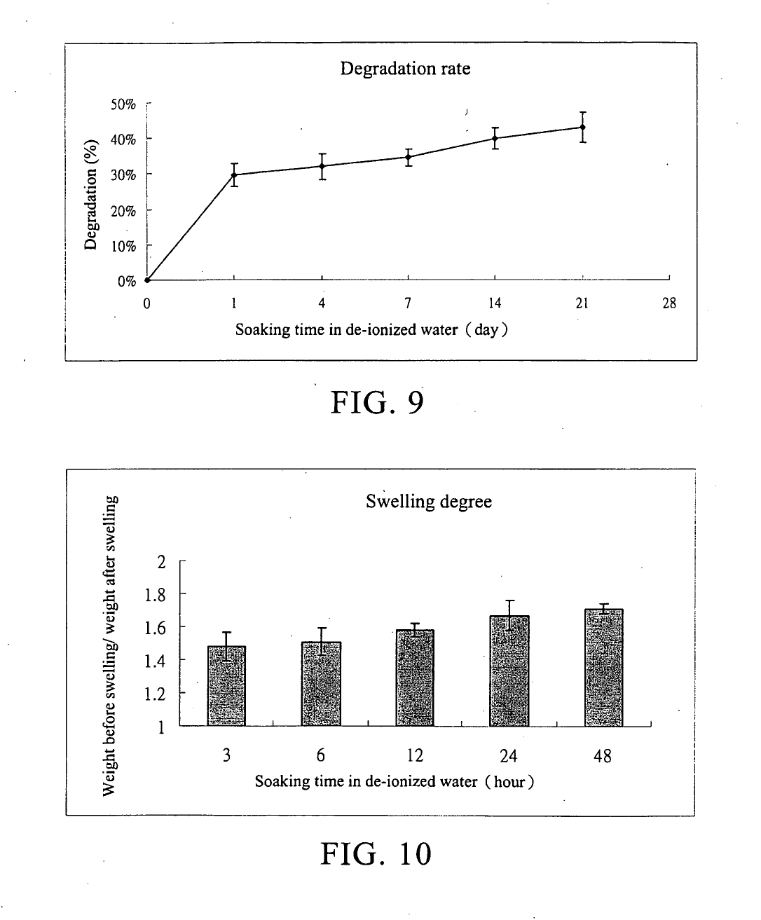

The dressing prepared in Example 2 was placed in deionized water, and the degradation rate thereof was observed. The result is shown in FIG. 9. As can be seen in FIG. 9, when the EGC active layer after the crosslink treatment by glutaraldehyde was immersed in the deionized water, the degradation rate thereof slowly increased from 30% in the first day to 32.5% in the fourth day, and from 35% in the seventh day gradually to 40% in the fourteenth day. The degradation rate in the twenty-first day was 42.5%.

This result showed that the structure of the EGC active layer was enhanced after the crosslink reaction, and when applied in wound treatment, it will not be rapidly decomposed by the body fluid, and its structure can be maintained for at least 21 days. Therefore, during the wound healing period, the dressing of the present invention can continuously support and promote the growth of fibroblast cells.

In another aspect, the swelling degree of the EGC active layer in the deionized water was tested. The result is shown in FIG. 10. As can be seen in FIG. 10, when the EGC active layer after the crosslink treatment with glutaraldehyde was immersed in the deionized water, the swelling degree thereof at the third hour increased to 1.5 times than its original weight; at the sixth hour slowly increased to 1.55 times; 1.6 times at the twelfth hour and gradually increased to 1.7 times at the twenty-fourth hour. At the forty-eighth hour, the swelling degree gradually becomes steady to 1.73 times.

The swelling degree test showed that the EGC active layer after the crosslink treatment, when applied in the wound therapy, can maintain its good structure, and the EGC active layer would not lose its function to mimic the extracellular matrix due to large variation of the fiber diameter (or the swelling degree). This makes fibroblast cells still adhere and grow successfully onto the structure of the EGC active layer during therapy.

### EXAMPLE 7

### The Water Contact Angle of the EGC Active Layer

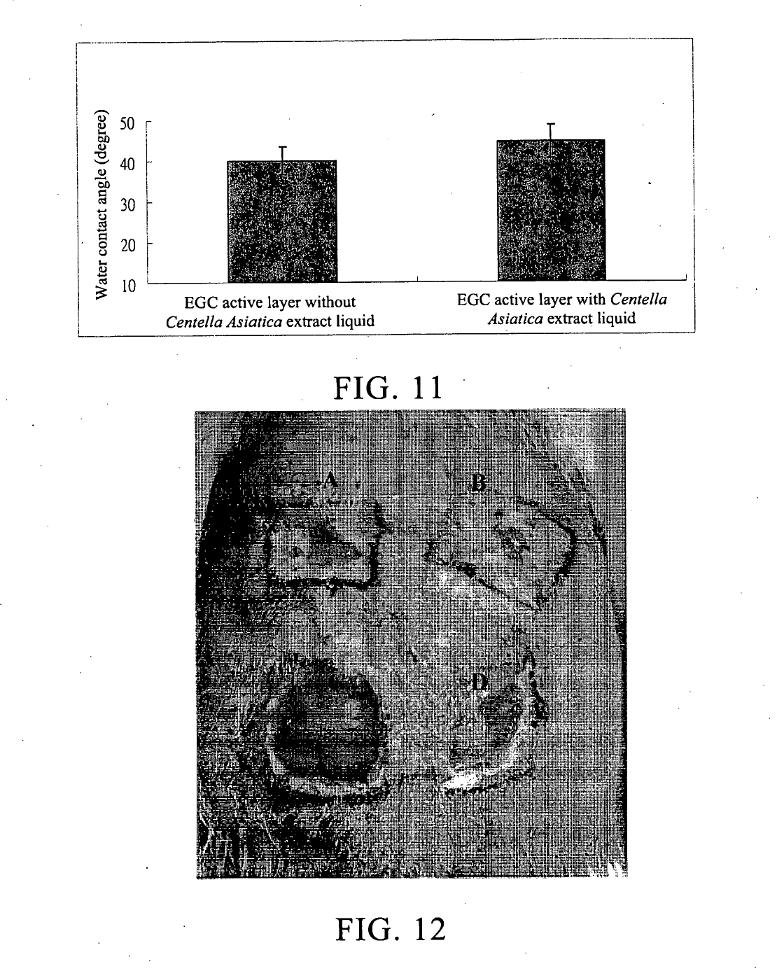

The water contact angle of the EGC active layer was measured and the results are shown in FIG. 11. As shown in FIG. 11, the water contact angle of the EGC active layer without being added with the Centella asiatica extract liquid was about 40±3 degree, and the water contact angle of the EGC active layer with the addition of the Centella asiatica extract liquid was about 44±4 degree. From the statistical analysis, there was no significant difference between the water contact angles of the active layers with or without the addition of the Centella asiatica extract liquid, and the angles were both less than 90 degree. Therefore, after the EGC active layer was treated with glutaraldehyde to conduct steam crosslinking for 45 minutes, the hydrophilic structure of the EGC active layer would not be affected.

### EXAMPLE 8

### Animal test

The dressing of the present invention was used to carry out an animal test of rat wound healing in this experiment. SD rats (purchased from the Biolasco Biotechnology Co., Ltd., Taiwan) were used. The experiment was divided into four groups: A—an experimental group: the EGC active layer comprising 46,800 μg/mL of the Centella asiatica extract liquid; B—a control group: the EGC active layer comprising no Centella asiatica extract liquid; C—a comparative group: a commercially available dressing; D—a blank group. The results are shown in FIG. 12.

FIG. 12 is a photo showing the wound recovery of the rat on day 14. Computing software was used to calculate the healing area of the rat skin regeneration to obtain the recovery rate of these four groups as follows: A—the experimental group: 88.68±0.82%; B—the control group: 83.96±1.70%; C—the comparative group: 34.30±1.24%; D—the blank group: 70.88±2.60% (i.e., A—the experimental group>B—the control group>D—the blank group>C—the comparative group. It was observed that the commercially available dressing of C—the comparative group had stickiness on the wound tissue, and the nascent tissue was damaged when the dressing was taken off, and thus the recovery rate thereof was only about 34%. D—the blank group without using any dressing but only with gauze cover could achieve 70% recovery rate by the rat's self-healing. A—the experimental group with the addition of the Centella asiatica extract liquid had the best recovery rate on the SD rat's skin wound and could achieve nearly 90% recovery rate. Even the dressing without being added with the Centella asiatica extract liquid (B—the control group) also achieved 83% recovery rate. This indicates that the material of the dressing of the present invention has excellent biocompatibility, which can help the proliferation of fibroblast cells, and with the addition of the Centella asiatica extract liquid, it can even speed up the growth of fibroblast cells. Therefore, the dressing of the present invention can promote accelerated wound healing to the tissue and achieve the effect of recovering the wound tissue in a short period of time.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A dressing, comprising:
(a) a substrate layer; and
(b) an active layer, which is a nanofiber layer and comprises:
(b1) at least one of gelatin and collagen;
(b2) polyvinyl alcohol (PVA); and
(b3) at least one of asiaticoside and a Centella asiatica extract.

2. The dressing as claimed in claim 1, wherein the substrate layer is a chitosan layer.

3. The dressing as claimed in claim 1, wherein the amount of ingredient (b1) in the active layer is about 80% to 99%, based on the total weight of ingredients (b1) and (b2).

4. The dressing as claimed in claim 1, wherein the active layer is formed onto a surface of the substrate layer by electrospinning.

5. The dressing as claimed in claim 4, wherein the active layer is formed by the following steps:
(I) providing a first solution comprising ingredient (b1);
(II) providing a second solution comprising ingredient (b2);
(III) mixing the first solution and the second solution to provide a third solution;
(IV) adding a fourth solution comprising ingredient (b3) into the third solution to obtain a fifth solution;
(V) electrospinning the fifth solution to form a fiber layer onto a surface of the substrate layer; and
(VI) carrying out a crosslink reaction to crosslink the fiber layer to form the active layer.

6. The dressing as claimed in claim 5, wherein in the third solution of step (III), the concentration of ingredient (b1) is about 130 to 170 mg/mL, and the concentration of ingredient (b2) is about 5 to 30 mg/mL; and in the fifth solution of step (IV), the amount of the third solution is about 60% to 95%, based on the total volume of the third solution and the fourth solution.

7. The dressing as claimed in claim 5, wherein in the third solution of step (III), the concentration of ingredient (b1) is about 140 to 160 mg/mL, and the concentration of ingredient (b2) is about 5 to 15 mg/mL; and in the fifth solution of step (IV), the amount of the third solution is about 80% to 90%, based on the total volume of the third solution and the fourth solution.

8. The dressing as claimed in claim 5, wherein in step (V), the fifth solution is introduced into an injector equipped with a needle to carry out the electrospinning to form the fiber layer on the substrate layer, and the electrospinning is operated for about 2 to 8 hours at conductions including a voltage from about 20 to 30 KV, a feeding flow rate of the fifth solution in the injector from about 0.005 to 0.015 mL/hour, and a distance between the needle head of the injector and the surface of the substrate layer from about 7 to 13 cm.

9. The dressing as claimed in claim 8, wherein the electrospinning is operated for about 3 to 5 hours at conductions that the voltage is about 24 to 26 KV, the feeding flow rate of the fifth solution in the injector is about 0.008 to 0.012 mL/hour and the distance between the needle head of the injector and the surface of the substrate layer is about 9 to 11 cm.

10. The dressing as claimed in claim 5, wherein in step (VI), an aqueous solution containing glutaraldehyde of about 40 to 60% by volume is used to carry out a steam crosslink reaction on the fiber layer for about 35 to 55 minutes.

11. The dressing as claimed in claim 5, wherein in step (VI), an aqueous solution containing glutaraldehyde of about 45 to 55% by volume is used to carry out a steam crosslink reaction on the fiber layer for about 40 to 50 minutes.

12. A method for preparing the dressing as claimed in claim 1, comprising:
(I) providing a first solution comprising ingredient (b1);
(II) providing a second solution comprising ingredient (b2);
(III) mixing the first solution and the second solution to provide a third solution;
(IV) adding a fourth solution comprising ingredient (b3) into the third solution to obtain a fifth solution;
(V) electrospinning the fifth solution to form a fiber layer onto a surface of the substrate layer; and
(VI) carrying out a crosslink reaction to crosslink the fiber layer to form the active layer.

13. The method as claimed in claim 12, wherein the substrate layer is a chitosan layer.

14. The method as claimed in claim 12, wherein the amount of ingredient (b1) in the active layer is about 80% to 99%, based on the total weight of ingredients (b1) and (b2).

15. The method as claimed in claim 12, wherein in the third solution of step (III), the concentration of ingredient (b1) is about 130 to 170 mg/mL, and the concentration of ingredient (b2) is about 5 to 30 mg/mL; and in the fifth solution of step (IV), the amount of the third solution is about 60% to 95%, based on the total volume of the third solution and the fourth solution.

16. The method as claimed in claim 12, wherein in the third solution of step (III), the concentration of ingredient (b1) is about 140 to 160 mg/mL, and the concentration of ingredient (b2) is about 5 to 15 mg/mL; and in the fifth solution of step (IV), the amount of the third solution is about 80% to 90%, based on the total volume of the third solution and the fourth solution.

17. The method as claimed in claim 12, wherein in step (V), the fifth solution is introduced into an injector equipped with a needle to carry out the electrospinning to form the fiber layer on the substrate layer, and the electrospinning is operated for about 2 to 8 hours at conductions including a voltage from about 20 to 30 KV, a feeding flow rate of the fifth solution in the injector from about 0.005 to 0.015 mL/hour, and a distance between the needle head of the injector and the surface of the substrate layer from about 7 to 13 cm.

18. The method as claimed in claim 17, wherein the electrospinning is operated for about 3 to 5 hours at conductions that the voltage is about 24 to 26 KV, the feeding flow rate of the fifth solution in the injector is about 0.008 to 0.012 mL/hour, and the distance between the needle head of the injector and the surface of the substrate layer is about 9 to 11 cm.

19. The method as claimed in claim 12, wherein in step (VI), an aqueous solution containing glutaraldehyde of about 40 to 60% by volume is used to carry out a steam crosslink reaction on the fiber layer for about 35 to 55 minutes.

20. The method as claimed in claim 12, wherein in step (VI), an aqueous solution containing glutaraldehyde of about 45 to 55% by volume is used to carry out a steam crosslink reaction on the fiber layer for about 40 to 50 minutes.
